(19) United States
(12) Patent Application Publication
(10) Pub. No.: US 2013/0204220 A1
(43) Pub. Date: Aug. 8, 2013

(54) DIAPER WITH A PLURALITY OF BABY WIPES

(71) Applicant: Greg Gunter, Okeechobee, FL (US)

(72) Inventor: Greg Gunter, Okeechobee, FL (US)

(21) Appl. No.: 13/368,295

(22) Filed: Feb. 7, 2012

(51) Int. Cl. A61F 13/84

(52) U.S. Cl. 604/385.06

(57) ABSTRACT

The present invention is a diaper with a plurality of baby wipes that includes an exterior diaper with a back side and a baby wipe pocket with a front with an opening that is disposed on the back side. The diaper also includes a peel-off strip that covers the opening and is peeled off when ready for use and a resealable fastener that is exposed when the peel-off strip is removed and is opened to retrieve one or more desired quantities of the baby wipes that are retrieved from the baby wipe pocket. The exterior diaper can be a standard foldable diaper or a pull-up diaper.

### TECHNICAL FIELD & BACKGROUND

Currently there are limited options for caregivers who desire to simplify and condense the multiple supplies required for infant care. Often, when traveling or when in a haste, individuals may find themselves lacking in sufficient cleaning wipes for adequate use during diaper changes.

The present invention generally relates to a diaper. More specifically, the invention is a diaper with a plurality of baby wipes.

It is an object of the invention to provide a diaper with wipes that has a packet of wipes disposed on or within the diaper.

It is an object of the invention to provide a diaper with wipes that has a packet of wipes that are resealable.

It is an object of the invention to provide a diaper with wipes that has a packet of wipes for a variety of different diaper types.

What is really needed is a diaper with wipes that has a packet of wipes disposed on or within the diaper that has a packet of wipes that are resealable and that has a packet of wipes for a variety of different diaper types.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a top view of a standard foldable diaper with a plurality of cleaning wipes, according to an embodiment of the present invention.

FIG. 2A illustrates a front view of a pull-up diaper with a plurality of cleaning wipes, according to an embodiment of the present invention.

FIG. 2B illustrates a front view of a pull-up diaper with a plurality of cleaning wipes, according to an embodiment of the present invention.

FIG. 2C illustrates a front view of a pull-up diaper with a plurality of cleaning wipes, according to an embodiment of the present invention.

FIG. 2D illustrates a front view of a pull-up diaper with a plurality of cleaning wipes, according to an embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is utilized repeatedly. The phrase generally does not refer to the same embodiment, however, it may. The terms “comprising”, “having” and “including” are synonymous, unless the context dictates otherwise.

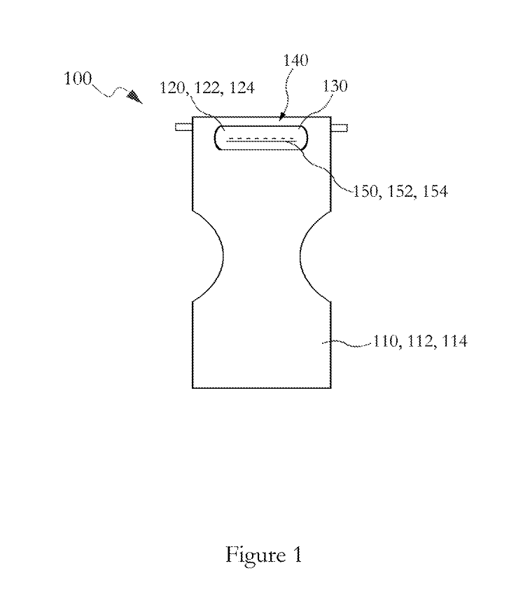

FIG. 1 illustrates a top view of a diaper with a plurality of cleaning wipes 100, according to an embodiment of the present invention.

The diaper with a plurality of cleaning wipes 100 includes an exterior diaper 110, a baby wipe pocket 120, a plurality of baby wipes 130, a peel-off strip 140 and a fastener 150. The exterior diaper 110 has a back side 112 and can be any type of diaper that can include a standard foldable diaper 114, which is the type of exterior diaper 110 illustrated in FIG. 1A, although other suitable types of diapers such as a swimming diaper (not shown) can be utilized as well. The baby wipe pocket 120 has a front 122 with an opening 124 and is air tight and can be disposed on the back side 112 of the exterior diaper 110 or can be disposed on any suitable location on the exterior diaper 114. The baby wipes 130 are contained in the baby wipe pocket 120. Typically there are 4 to 6 baby wipes 130 that are contained in the baby wipe pocket 120, although any suitable number of baby wipes 130 can be contained in the baby wipe pocket 120. The peel-off strip 140 covers the opening 124 of the baby wipe pocket 120 and can be peeled off when ready for use. The fastener 150 is typically a hook and loop fastener 152 and is resealable although other suitable types of fasteners can also be utilized such as a tongue and groove fastener 154. Once the strip 140 is removed, the fastener 150 can be exposed and opened and one or more desired quantities of baby wipes 130 can be retrieved from the baby wipe pocket 120. The baby wipe pocket 120 can be resealed as desired as well with the fastener 150.

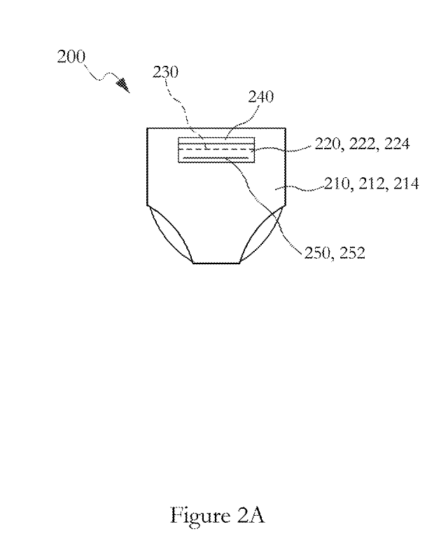

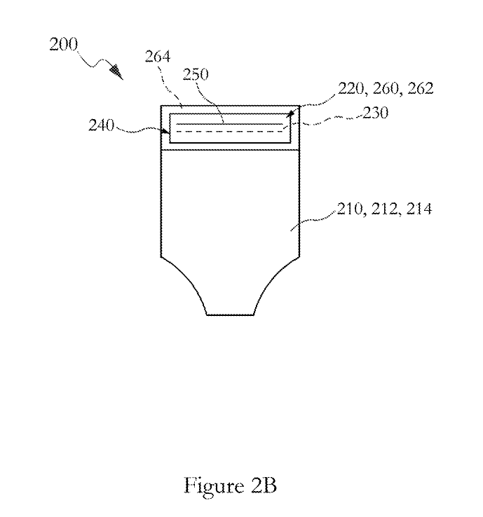

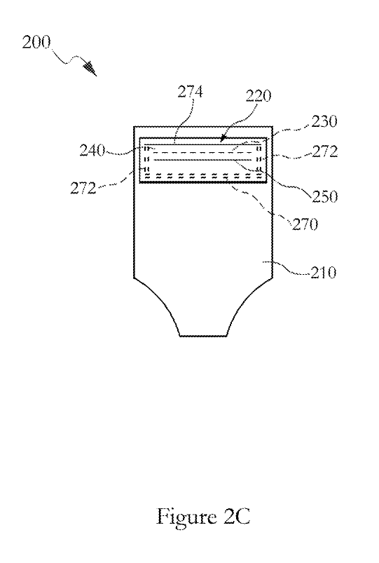

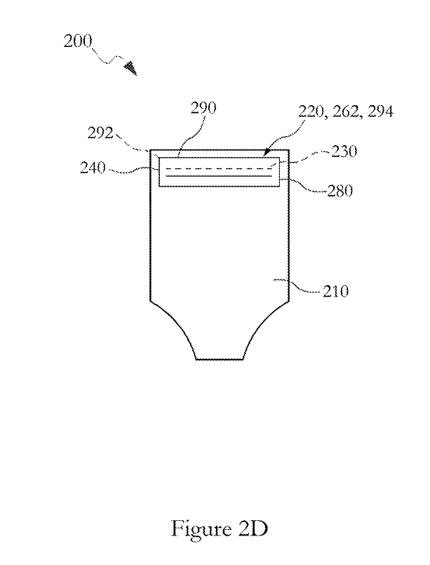

FIG. 2A illustrates a front view of a diaper with a plurality of cleaning wipes 200, according to an embodiment of the present invention. The diaper with cleaning wipes 200 includes similar features to the diaper with cleaning wipes 100 illustrated in FIG. 1 that include an exterior diaper 210, a baby wipe pocket 220, a plurality of baby wipes 230, a peel-off strip 240 and a fastener 250. The exterior diaper 210 has a back side 212 and can be any type of diaper that can include a pull-up diaper 214, which is the type of exterior diaper 210 illustrated in FIG. 2A. Similar to the diaper with cleaning wipes 100 illustrated in FIG. 1, the baby wipe pocket 220 has a front 222 with an opening 224 and is air tight and can be disposed on the back side 212 of the exterior diaper 210 or can be disposed on any suitable location on the exterior diaper 214. The baby wipes 230 are contained in the baby wipe pocket 220. Typically there are 4 to 6 baby wipes 230 that are contained in the baby wipe pocket 220, although any suitable number of baby wipes 230 can be contained in the baby wipe pocket 220. The peel-off strip 240 covers the opening 224 of the baby wipe pocket 220 and can be peeled off when ready for use. The fastener 250 is typically a hook and loop fastener 252 and is resealable although other suitable types of fasteners can also be utilized. Once the strip 240 is removed, the fastener 250 can be exposed and opened and one or more desired quantities of baby wipes 230 can be retrieved from the baby wipe pocket 220. The baby wipe pocket 220 can be resealed as desired as well with the fastener 250.

FIG. 2B illustrates a front view of a pull-up diaper with a plurality of cleaning wipes 200, according to an embodiment of the present invention. The diaper with cleaning wipes 200 includes similar features to the diaper with cleaning wipes 100 illustrated in FIG. 1 that include an exterior diaper 210, a baby wipe pocket 220, a plurality of baby wipes 230, a peel-off strip 240 and a fastener 250. The exterior diaper 210 has a back side 212 and can be any type of diaper that can include a pull-up diaper 214, which is the type of exterior diaper 210 illustrated in FIG. 2B. The exterior diaper 210 has a baby wipe pocket 220 that is prepackaged 260 with a bag of baby wipes 262 enclosed in the baby wipe pocket 220. There is also an opening 264 behind the baby wipe pocket 220 that a user can access and retrieve one or more baby wipes 230.

FIG. 2C illustrates a front view of a pull-up diaper with a plurality of cleaning wipes 200, according to an embodiment of the present invention. The diaper with cleaning wipes 200 includes similar features to the diaper with cleaning wipes 100 illustrated in FIG. 1 that include an exterior diaper 210, a baby wipe pocket 220, a plurality of baby wipes 230, a peel-off strip 240 and a fastener 250. The exterior diaper 210 has a back side 212 and can be any type of diaper that can include a pull-up diaper 214, which is the type of exterior diaper 210 illustrated in FIG. 2C. In contrast to the pull-up diaper with a plurality of cleaning wipes 200 illustrated and described in FIGS. 2A and 2B, the baby wipe pocket 220 is laminated to the exterior diaper 210 on a bottom side 270, and two sides 272 and can be opened at a top side 274 as previously described in FIG. 2A and its description.

FIG. 2D illustrates a front view of a pull-up diaper with a plurality of cleaning wipes 200, according to an embodiment of the present invention. The diaper with cleaning wipes 200 includes similar features to the diaper with cleaning wipes 100 illustrated in FIG. 1 that include an exterior diaper 210, a baby wipe pocket 220, a plurality of baby wipes 230, a peel-off strip 240 and a fastener 250. The exterior diaper 210 has a back side 212 and can be any type of diaper that can include a pull-up diaper 214, which is the type of exterior diaper 210 illustrated in FIG. 2C. In contrast to the pull-up diaper with a plurality of cleaning wipes 200 illustrated and described in FIGS. 2A and 2B, the baby wipe pocket 220 contains a bag of baby wipes 262 similar to the bag of baby wipes 262 described in FIG. 2B, but is instead folded while contained in the baby wipe pocket 220. The folded bag of baby wipes 262 are covered by a sheet of material 280 with hook and loop material 290 that contains the folded bag of baby wipes 262 pressed against hook and loop material 292 disposed on the type of exterior diaper 210 forming a releasable baby wipe pocket 294 that can be grasped and separated to release the folded bag of baby wipes 262.

The diaper with a plurality of cleaning wipes are traditional diapers designed with an attached wipe containing bag, that introduces a modern alternative to ensuring ample supplies. Each diaper possesses a plastic air-tight holding bag affixed to the back exterior of the diaper, containing within four to six soft cloth baby wipes that can be easily retrieved via the zip-lock or tongue and groove fastener or other suitable fastener at the top of the bag. When in the process of cleaning a solid or liquid excretion from a baby or infant, a user simply utilizes the wipes provided with each covering, quickly and effectively, collecting the waste and then disposing of the diaper by placing all items into the former diaper's holding bag, thereby alleviating additional clean-up and the experience of lasting pungent odors. The diaper with a plurality of cleaning wipes can be marketed in various colors and styles to accommodate diverse consumers. The diaper with a plurality of cleaning wipes may be extended to include pull-up training pants and other common children's potty alternatives used to aide growing children. The feature of a combination diaper and cleaning cloth body covering removes the concern of forgetting or misplacing any wipes while simultaneously serving as a tactful method to performing hygienic maintenance.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A diaper with a plurality of baby wipes, comprising:
an exterior diaper with a back side;
a baby wipe pocket with a front with an opening that is disposed on said back side;
a peel-off strip that covers said opening and is peeled off when ready for use; and
a resealable fastener that is exposed when said peel-off strip is removed and is opened to retrieve one or more desired quantities of said baby wipes that are retrieved from said baby wipe pocket.

2. The diaper according to claim 1, wherein said baby wipes are contained in said baby wipe pocket.

3. The diaper according to claim 2, wherein said baby wipe pocket contains 4 to 6 said baby wipes.

4. The diaper according to claim 1, wherein said exterior diaper is a standard foldable diaper.

5. The diaper according to claim 1, wherein said exterior diaper is a pull-up diaper.

6. The diaper according to claim 1, wherein said fastener is a hook and loop fastener.

7. The diaper according to claim 1, wherein said fastener is a tongue and groove fastener.

8. A diaper with a plurality of baby wipes, comprising:
an exterior diaper with a back side;
a baby wipe pocket with a front, a bottom side, a top side and two sides with an opening that is disposed on said back side, said baby wipe pocket is prepackaged with a bag of baby wipes enclosed in said baby wipe pocket;
a peel-off strip that covers said opening and is peeled off when ready for use; and
a resealable fastener that is exposed when said peel-off strip is removed and is opened to retrieve one or more desired quantities of said baby wipes that are retrieved from said baby wipe pocket.

9. The diaper according to claim 8, wherein said baby wipes are contained in said baby wipe pocket.

10. (canceled)

11. The diaper according to claim 9, wherein said diaper has an opening behind said baby wipe pocket to access and retrieve said one or more baby wipes.

12. The diaper according to claim 9, wherein said baby wipe pocket is laminated to said exterior diaper.

13. The diaper according to claim 12, wherein said baby wipe pocket is laminated to said bottom side and said two sides.

14. The diaper according to claim 13, wherein said baby wipe pocket is opened on said top side.

15. The diaper according to claim 9, wherein said bag of baby wipes are covered by a sheet of material with hook and loop material disposed on said sheet of material that is pressed against hook and loop material disposed on said exterior diaper forming a releasable baby wipe pocket.

16. The diaper according to claim 15, wherein said releasable baby wipe pocket is grasped and separated to release said bag of baby wipes.

17. The diaper according to claim 9, wherein said baby wipe pocket contains 4 to 6 said baby wipes.

18. The diaper according to claim 8, wherein said exterior diaper is a pull-up diaper.

19. The diaper according to claim 8, wherein said fastener is a hook and loop fastener.

20. The diaper according to claim 8, wherein said fastener is a tongue and groove fastener.
